(19) United States
(12) Patent Application Publication
(10) Pub. No.: US 2020/0250554 A1
(43) Pub. Date: Aug. 6, 2020

(54) METHOD AND STORAGE MEDIUM FOR PREDICTING THE DOSAGE BASED ON HUMAN PHYSIOLOGICAL PARAMETERS

(71) Applicant: Jabil Circuit (Shanghai) Co., Ltd., Shanghai (CN)

(72) Inventors: Jiawei Shao, Shanghai (CN); Yinong Zhao, Shanghai (CN)

(73) Assignee: Jabil Circuit (Shanghai) Co., Ltd., Shanghai (CN)

(21) Appl. No.: 16/778,182

(22) Filed: Jan. 31, 2020

(30) Foreign Application Priority Data
Feb. 1, 2019 (CN) 201910105012.3

(51) Int. Cl. G06N 5/04; G06N 5/00; G16H 50/70; G16H 20/17; G06F 16/25

(52) U.S. Cl. CPC G06N 5/04; G06N 5/003; G06F 16/254; G16H 20/17; G16H 50/70

(57) ABSTRACT

Described herein is a method and storage medium for predicting the dosage based on human physiological parameters, which can effectively predict the dosage for a patient based on the physiological parameters given by the patient. The method includes obtaining dosage data and multiple human physiological parameter data of multiple testers as raw data, preprocessing the raw data to obtain input data as a training set, based on the input data, establishing a decision tree by classification and regression tree algorithm, which includes generating the decision tree based on the feature extraction of the input data, and pruning the generated decision tree and selecting the optimal sub-tree by using a validation data set, inputting a user's human physiological parameter data, and predicting the required dosage according to the established decision tree.

### CROSS REFERENCE TO RELATED APPLICATION

The present application claims priority to Chinese Patent Application No. 201910105012.3 filed Feb. 1, 2019, incorporated herein in its entirety.

### TECHNICAL FIELD

The present invention relates to the field of artificial intelligence, in particular to a method and storage medium for predicting the dosage based on human physiological parameters.

### BACKGROUND

In the future medical field, the application of computer technology will play an increasingly important role, and machine learning as an implementation of artificial intelligence is highly sought after. Machine learning helps people use a large amount of existing data for analysis, inference, and prediction, so that the services provided by medical devices are closer to the objective reality and more in line with the needs of modern customers.

For example, in the treatment of asthma, inhaling medicine through an inhalation device such as an inhalation box is a commonly used treatment for asthma. In order to treat asthma more effectively, people have tried to explore the relationship between the medicine inhaling volume and various human physiological parameters.

However, the information transmitted by medical devices is extremely large in both attributes and elements. Specifically, data is obtained through two hardware devices (an inhalation box and a physiological detection device). But the data transmitted by these two hardware devices contains extremely high dimensions, for example, there may be as many as 26 dimensions. Therefore, it is difficult to directly obtain whether there is a relationship between the data, or what kind of relationship exists. That is, because the data is huge and cluttered, it is difficult to determinate the relationship between the medicine inhaling volume obtained from the inhalation box and various human physiological parameters obtained from the physiological detection device. Not only for the treatment of asthma, but also for the treatment of other diseases, we also hope to find the relationship between the dosage and various human physiological parameters.

### SUMMARY

In view of the above problems, the technical problem solved is to provide a method and storage medium for predicting the dosage based on human physiological parameters, which can effectively predict the dosage for a patient based on the physiological parameters given by the patient.

In implementations, a method for predicting the dosage based on human physiological parameters comprises obtaining dosage data and multiple human physiological parameter data of multiple testers as raw data; preprocessing the raw data to obtain input data as a training set; based on the input data, establishing a decision tree by classification and regression tree algorithm, which includes: generating the decision tree based on the feature extraction of the input data, and pruning the generated decision tree and selecting the optimal sub-tree by using a validation data set; inputting a user's human physiological parameter data, and predicting the required dosage according to the established decision tree.

In implementations, effectively obtaining the relationship between the dosage (medicine inhaling volume data) and human physiological parameters is available, so that the dosage can be effectively predicted for a patient based on the physiological parameters given by the patient.

In implementations, the method further comprising using generalized regression neural network to post-optimize the output of the decision tree.

In implementations, the method further comprising using BADT to process null data specifically to post-optimize the output of the decision tree.

In implementations, the generation of the decision tree uses the Gini index to select the optimal feature, and to determine the optimal splitting point of the feature at the same time.

In implementations, the pruning comprising cutting off the sub-trees continuously from the bottom of the complete tree form of the decision tree; testing the sequence of the sub-trees on the independent verification data set by the cross-validation method, from which the optimal sub-tree is selected.

In implementations, the preprocessing comprising correlating the dosage data with the human physiological parameters on the time axis.

In implementations, the preprocessing further comprising processing the input data by ETL, and processing the output data of the decision tree by ETL again as input data, thereby continuously iterating.

In implementations, a storage medium stores instructions that can be executed by a computer device and can be read by the computer device; the instructions cause the computer device to perform the following steps: obtaining dosage data and multiple human physiological parameter data of multiple testers as raw data; preprocessing the raw data to obtain input data as a training set; based on the input data, establishing a decision tree by classification and regression tree algorithm, which includes: generating the decision tree based on the feature extraction of the input data, and pruning the generated decision tree and selecting the optimal sub-tree by using a validation data set; receiving a user's human physiological parameter data, and predicting the required dosage according to the established decision tree.

The foregoing and other objects, features, and advantages of the present invention will be better understood based on the following detailed embodiments and with reference to the accompanying drawings.

### BRIEF DESCRIPTION OF THE DRAWINGS

FIG. 1 is a basic flowchart of a method for predicting the dosage based on human physiological parameters according to an embodiment of the present invention;

FIG. 2 is a schematic flowchart of a classification and regression tree algorithm;

FIG. 3 is a schematic flowchart of using generalized regression neural network to post-optimize the output of the decision tree;

FIG. 4A-FIG. 4B are schematic flowcharts of using BADT to process null data specifically to post-optimize the output of the decision tree;

FIG. 5 is a schematic diagram of data preprocessing;

FIG. 6 is an exemplary diagram of data source object according to an embodiment of the present invention;

FIG. 7 is an example of information types of the sample data;

FIG. 8 is an example of input data after data preprocessing;

FIG. 9 is an example of the data after performing decision tree algorithm once;

FIG. 10 is the result of comparison between the predictive result of the decision tree algorithm and the actual inhaling volume;

FIG. 11 is an example histogram of the data after performing the decision tree algorithm 100 times;

FIG. 12 is an example matrix of the data after performing the decision tree algorithm 100 times;

FIG. 13 is a schematic diagram of the basic architecture of the generalized regression neural network;

FIG. 14 is an example of the data after GRNN and BADT optimization;

FIG. 15 is a comparison diagram between the predictive result and the actual test;

FIG. 16 is a comparison diagram of accuracy of the predictive results;

FIG. 17 is a comparison diagram of optimization algorithms;

FIG. 18 is a schematic diagram of the decision tree.

### DETAILED DESCRIPTION

The present invention is further described below with reference to the accompanying drawings and the following embodiments. It should be understood that the drawings and the following embodiments are only used to illustrate the present invention, but not to limit the present invention.

In order to be able to effectively predict the dosage for a patient based on the physiological parameters given by the patient, the present invention provides a method for predicting the dosage based on human physiological parameters. In the following embodiments, the treatment of asthma will be described in detail as an example. However, the present invention is not limited to this, and can also be applied to determinate the relationship between the dosage for other diseases and the human physiological parameters.

In an embodiment of the present invention, use the inhalation box and the physiological detection device respectively to obtain the medicine inhaling volume data and the multiple human physiological parameters data as the raw data, which is the information without any manual preprocessing. In this embodiment, as an example, the following 26 human physiological parameters are selected: weight; Heart_Rate_Variabiliy_LF (Sympathetic and parasympathetic nerve activity indicators; MAP (Mean arterial pressure; Systolic; Systolic_PTT (PTT means Pulse transit time); Heart_Rate_Variability_HF (Parasympathetic nerve activity indicators); PTT_Raw; Age; Diastolic_PTT (Diastolic pulse transfer time); Height; RR_Interval (R-R interval of electrocardiogram); Classification_Arousal (Brainwave classification in consciousness (beta wave classification); Heart_Rate_Curve; Diastolic; Sympatho_Vagal_Balance; Sleep_Wake; Gender; Activity (Original value of activity level, high indicates user's activity level); SpO₂:Cardio_rhythm (Heart rate analysis, such as arrhythmia, tachycardia, bradycardio; Acti_Profile (Activity levels based on Activity and predefined scopes, such as Low Acti, Median Acti, High Acti); Autonomic_Arousals (Pulse index of pleth); Cardio_complex (Related results of tachycardia (narrow QRS complex); Systolic_events (Systolic Rise, correlation analysis of ECG waveform); PTT_Events (Rising or falling state of PTT and its time interval); and Position (Such as Prone, Upright, Left, Right, Upright, Supine, Run).

Although this embodiment uses the above-mentioned 26 physiological parameters, the implementations are not limited to this, the quantity and parameter types can be changed. For example, other physiological parameters such as BMI (body mass index) and so on can be used.

Because there are many dimensions of physiological parameters, for example, the above-mentioned multiple physiological parameters have as many as 26 dimensions, it is difficult to obtain the relationship between the physiological parameter data and the medicine inhaling volume data directly. To this end, the inventors continue to study and try to achieve the following objectives: to investigate the relationship between various physiological parameters and medicine inhaling volume, which of the physiological parameters is most relevant to the inhaling volume (weight), and is it possible to predict inhaling volume based on a given physiological parameter. In order to achieve the above-mentioned objectives, the implementations use a machine learning method to analyze the data, that is, the decision tree algorithm in machine learning.

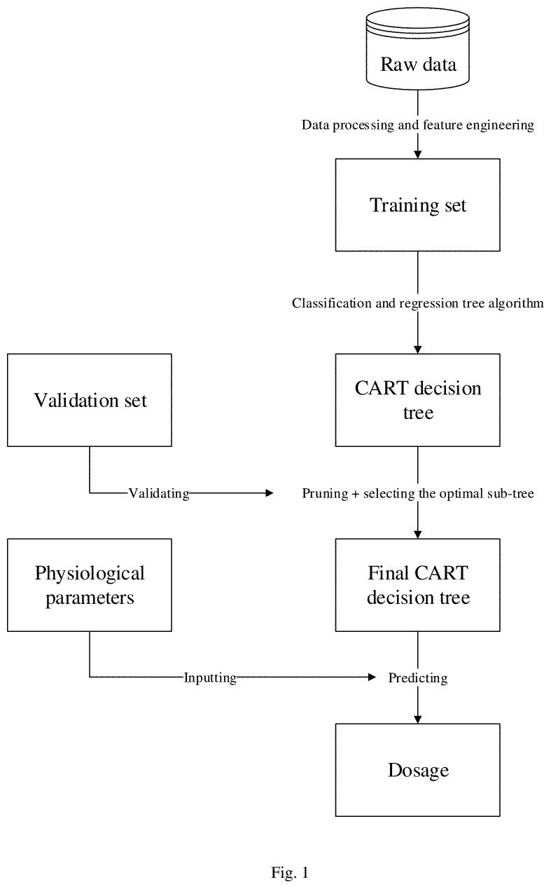

To this end, the implementations provide a method for predicting the dosage based on human physiological parameters, as shown in FIG. 1, the method comprising the following steps: obtaining dosage data and multiple human physiological parameter data of multiple testers as raw data; preprocessing the raw data to obtain input data as a training set; based on the input data, establishing a decision tree by classification and regression tree algorithm, which includes: generating the decision tree based on the feature extraction of the input data, and pruning the generated tree and selecting the optimal sub-tree by using a validation data set; inputting a user's human physiological parameter data, and predicting the required dosage according to the established decision tree.

According to implementations, the relationship between the dosage (medicine inhaling volume data) and the human physiological parameters can be effectively obtained, so that the dosage for the user can be effectively predicted based on the physiological parameters given by the user.

The method is described in further detail below.

<Selecting Prediction Model>

In fact, current technology has many algorithms that can perform predictive analysis and feature extraction, but there are several problems to be faced when processing and analyzing data: first, the process of extracting features is separate from the process of prediction; second, the rules of data processing are not directly understood by people, there are some abstract and complicated mathematical formulas; third, the preprocessing of the data is very troublesome, especially in the case of a large amount of data, the workload for normalization, null value, and missing value processing is relatively large.

To this end, the implementations use Classification and Regression Tree (CART) algorithm. “Classification” focuses on the feature identification and feature extraction of the data, and “Regression” focuses on determining the predicted probability distribution in the feature's division units. The algorithm can be used for unified extraction, prediction, its judgment rules is relatively easy to understand, and the data preprocessing is less. The details are as follows.

<Classification and Regression Tree (CART)>

CART is a method of outputting the conditional probability of random variables under the conditions of the given input data (the so-called input data is the training set of the raw data after data processing and feature engineering processing, the input data can be a parameter matrix, in one example, it can be a 890*27 matrix, including 26 physiological parameters and BMI). CART, taking the decision tree is a binary tree as an example, the node feature values of the tree are “yes” and “no” (for example, the left branch is “yes” and the right branch is “no”). In this way, recursing upward continuously from leaf node at the bottom layer, the decision tree is equivalent to recursively binarizing each feature.

Classification and regression tree is mainly divided into two steps: 1) Generation of the tree: generating a decision tree based on the feature extraction of the training set (which is the input data). In other words, after performing the classification and regression tree algorithm operation on the input data, a CART decision tree is obtained.

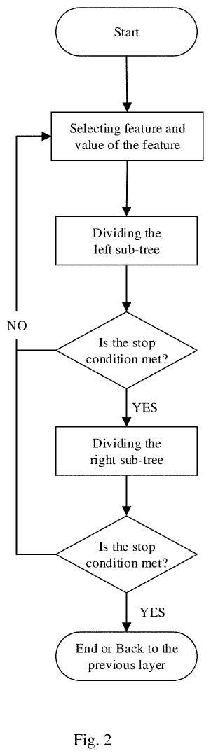

FIG. 2 is a schematic flowchart of a classification and regression tree algorithm. The following describes the steps of generating a decision tree with a specific example in conjunction with FIG. 2.

For example for the following raw data:

| | | | Mean arterial | Medicine |
|---|---|---|---|---|
| Test patient | Weight | Height | pressure | inhaling volume |
| D1 | 49 | 150 | 85 | 25 mg |
| D2 | 75 | 170 | 90 | 50 mg |
| D3 | 100 | 200 | 95 | 100 mg |
| D4 | 90 | 185 | 85 | 90 mg |

1. Select the optimal splitting variable j and the optimal splitting point s (which is the feature and feature value selection step shown in FIG. 2). The first variable in this data set is weight, so weight is preferred as the optimal splitting variable.
1.1 Calculate the optimal splitting point for the variable “weight”: Because the range of weight is 49 to 100, and there are 4 samples, so select t=(100−49)/(4−1)=12.75 as the spacing of the splitting interval, considering 4 splitting interval sections: [49, 49+t], [49+t, 49+2*t], [49+2*t, 49+3*t], [49+3% 100]
Loss function defined as a squared loss function: Loss (y, f(x))=(f(x)−y)², select the optimal splitting variable j and the optimal splitting point s, solve the following formula M to minimize its value:

$${min}_{j , s} [ {min}_{{c}_{1}} {\sum }_{{x}_{1} ∈ {R}_{1} ( j , s )} {( {y}_{i} - {c}_{1} )}^{2} + {min}_{{c}_{2}} {\sum }_{{x}_{1} ∈ {R}_{1} ( j , s )} {( {y}_{i} - {c}_{2} )}^{2} ]$$

In the formula, Cₘ=ave(yᵢ|xᵢ∈Rₘ)
1.1.1 Take the first splitting point s1=49+1*t, that is, the first splitting interval is [49, 49+12.75], the second splitting interval is [61.75, 100], this splitting point divides 4 samples into two parts: R1={49}, R2={75, 90, 100}.
1.1.1.1 Calculate c1=25, c2=(50+90+100)/3=80
and get the following table:

| | | | |
|---|---|---|---|
| S | 49 + | 49 + | 49 + |
| | 12.75 = 61.75 | 2*12.75 = 74.5 | 3*12.75 = 87.25 |
| R1 | {49} | {49} | {49, 75} |
| R2 | {75, 90, 100} | {75, 90, 100} | {90, 100} |
| c1 | 25 | 25 | 37.5 |
| c2 | 80 | 80 | 95 |

1.1.1.2 Substitute c1, c2 into formula M, and calculate the left part of formula M corresponding to s1:

$${min}_{{c}_{1}} {\sum }_{{x}_{1 ∈} {R}_{1 ( j , s )}} {( {y}_{i} - {c}_{1} )}^{2}$$

M1=(25−25)\hat{ }2=0
Calculate the right part of formula M corresponding to s1

$${min}_{{c}_{2}} {\sum }_{{x}_{1} ∈ {R}_{2} ( j , x )} {( {y}_{i} - {c}_{2} )}^{2}$$

$$M 2 = ( 50 - 80 ) \hat{} 2 + ( 100 - 80 ) \hat{} 2 + ( 90 - 80 ) \hat{} 2 = 900 + 400 + 100 = 1400$$

Then the value of formula M corresponding to s1 is m1=0+1400=1400.
1.1.2 Calculate the value of M (m2, m3) corresponding to s2, s3, and get values of all the M:

| | | | |
|---|---|---|---|
| S | 61.75 | 74.5 | 87.25 |
| R1 | {49} | {49} | {49, 75} |
| R2 | {75, 90, 100} | {75, 90, 100} | {90, 100} |
| c1 | 25 | 25 | 37.5 |
| c2 | 80 | 80 | 95 |
| M | 1400 | 1400 | 362.5 |

1.1.3 According to the table above, when s=87.25, the minimum value of M is 362.5. Therefore, for the splitting variable “weight”, select the splitting point 87.25.
1.1.4 Divide the area with the selected splitting point 87.25, the two areas are: R1={49, 75}, R2={90, 100}. c1=37.5, c2 =95 are correspond to the splitting point.
1.2 Calculate the optimal splitting point for the second variable “height”, since the calculation method is similar to the calculation of “weight”, here will not go into detail.
1.3 Compare the values of formulas M of “weight” and “height”, it can be calculated that when the first splitting variable is selected as “weight”, a smaller value of formula M can be obtained, so:
   The first optimal splitting variable is “weight”.
   The first optimal splitting point is: weight=87.25.
   The first splitting point divides the area into two parts: R1={49, 75}, R2={90, 100}, and the corresponding output values of the decision tree are c1=37.5, c2=95.
2. Continue recursively calling step 1 on R1={49, 75} which is selected in step 1, and obtain the optimal splitting variable and optimal splitting point for R1, here will not go into detail.
3. Generate a regressing tree:
Keep recursing until the stop condition is met (each R is indivisible).

In the process of calculating the decision tree, the left and right sub-trees must be recursively divided continuously until the entire decision tree is generated. This process is to continuously find the optimal splitting variable (that is, which variable should be used for splitting) and the splitting point (which value should be used as divide for splitting).

The purpose of seeking j and S is to further split the current sub-tree more reasonably. Suppose that the splitting variable j “height” and the splitting point S1=61.75 are currently being calculated: R represents a temporary array which composed by values of height of the entire current sub-tree after sorting.

Splitting point S1 divides the array into two parts, R1 represents the left sub-array, that is, the array composed by values of height j which are less than the splitting point; R2 represents the right sub-array, that is, the array composed by values of height j which are greater than or equal to the splitting point. C1 represents the average value of the left sub-array of R for the splitting point S1; C2 represents the average value of the right sub-array.

Values of M consist two parts, among them: M1 is the sum of the variances of inhaling volume y and C1 for the splitting point S1. It can be understood as the splitting error effect of the left part after splitting R by the current splitting point S1. Similarly, M2 represents the splitting error effect of the right part after splitting R by the splitting point S1. M=M1+M2 represents the total error of the left and right parts. We want the error to be the smallest, so we need to calculate the value of M for each splitting point in turn to minimize the error, then the current splitting point s is regarded as the optimal splitting point for the height variable j.

Generation of the classification tree uses Gini index to select the optimal feature, and determine the optimal binary splitting point of the feature at the same time;

in the process of classification, assuming that there are K classes, and the probability that the sample points belong to the kth class is pₖ, then the Gini index of the probability distribution is defined as:

$$Gini ( p ) = {\sum }_{k = 1}^{K} {p}_{k} ( 1 - {p}_{k} ) = 1 - {\sum }_{k = 1}^{K} {p}_{k}^{2}$$

For the binary classification problem, if the probability that the sample points belong to the first class is p, then the Gini index of the probability distribution is:

Gini(p)=2p(1−p)

For a given sample set D, its Gini index is:

$$Gini ( D ) = 1 - {\sum }_{k = 1}^{K} {( \frac{\vert {C}_{k} \vert}{\vert D \vert} )}^{2}$$

In the formula, cₖ is the sample subset in D which belong to the kth class, K is the number of classes. If sample set D is divided into two parts D₁ and D₂ according to whether feature A takes a possible value a, that is:

D₁={(x,y)∈D|A(x)=a}, D₂=D−D₁

Then under the condition of feature A, the Gini index of set D is defined as:

$$Gini ( D , A ) = \frac{\vert {D}_{1} \vert}{\vert D \vert} Gini ( {D}_{1} ) + \frac{\vert D 2 \vert}{\vert D \vert} Gini ( {D}_{2} )$$

The Gini index Gini(D) represents the uncertainty of set D, and the Gini index Gini(D,A) represents the uncertainty of set D after A=a segmentation. The larger the Gini index, the greater the uncertainty of the sample set.

According to the training data set, starting from the root node, recursively perform the following operations on each node to build a binary decision tree:
(1) Set the training data set of the node as D, calculate the Gini coefficient of the current feature to the data set. At this time, for each feature A, for each value a that is possible to obtain, divide D into two parts D₁ and D₂ according to whether the sample point test A=a is “yes” or “no”, and calculate the Gini index when A=a;
(2) Among all possible features A and all their possible splitting points a, the feature with the smallest Gini index and its corresponding splitting point are selected as the optimal feature and optimal splitting point, according to the optimal feature and optimal splitting point, generate two sub-nodes from the current node, and assign the training data set to the two sub-nodes according to features.
(3) Recursively call (1) and (2) on the two sub-nodes, until the stop condition is met.
(4) Generate the CART decision tree.

The condition that the algorithm stops calculating is that the number of samples in the node is less than the predetermined threshold, or the Gini index of the sample set is less than the predetermined threshold (the samples basically belong to the same class), or there are no more features.

2) Pruning of the tree: Pruning the generated tree and selecting the optimal sub-tree by using a validation data set, at this time, set the minimum loss function as the standard for pruning. The validation data set can be, for example, 100 new 890*27 matrix data obtained for training after re-sampling 890*27 input matrix data obtained from the raw data, these data cover about 63.2% of the original input data, the actual inhaling volume data corresponding to the remaining 36.8% of the matrix data can be used as verification data. These verification data ultimately come from the collection of the medicine box. Resampling is used to solve the problem of the imbalance of classification. This kind of situation happens because machine learning algorithms are usually designed to improve accuracy by reducing errors. So they do not consider the distribution/proportion of the classification or the balance of the classification. This embodiment uses the Bootstrap Aggregating algorithm to achieve the resampling process.

CART's pruning cuts off the sub-trees continuously from the bottom of the complete tree form of the decision tree, making the decision tree continuously smaller and better, so as to improve the accuracy of prediction.

The CART pruning algorithm consists of two steps: first, continuously pruning from the bottom of the decision tree T⁰ generated by the generation algorithm to the root node of the tree T₀ to form a subsequence [T₀, T₁, . . . , Tₙ]; then testing the sequence of sub-trees on the independent verification data set by the cross-validation method, and the optimal sub-tree is selected from it.

Record the tree generated by CART as TO, then start pruning from the bottom of TO to the root node. In the process of pruning, calculate the loss function: Cₙ(T)=C(T)+α[T], C(T) is the prediction error of the training data, [T] is the complexity of the model.

For a fixed α, there must be a tree T_{α} in T0 to minimize the loss function C_{α}(T). That is, for each fixed α, there is a corresponding tree that minimizes the loss function. In this way, different a will generate different optimal tree, and we do not know which one is the best among these optimal trees, so we need to divide α into a series of areas in its value space, take one α in each area and obtain the corresponding optimal tree, finally select the optimal tree with the smallest loss function.

<Results of the Initial Stage>

After performing the decision tree algorithm once, we obtain a 26*2 matrix, 26 is the number of all the attribute features of the information, and 2 represents the attribute name and weight index. The data is arranged in descending order of weights, the larger the weight, the more important the attribute is, that is, the greater the positive correlation between the attribute and the medicine inhaling volume, the matrix is shown in FIG. 9.

It is not difficult to see from FIG. 9 that the weight index of weight is the highest, which means that weight is the most important of all attributes and has the greatest influence on the medicine inhaling volume. In addition, PTT_Raw and MAP have a relatively great degree of influence, but their weight indexes are far less than weight's on the order of magnitude. It can be preliminarily considered that weight is the most important parameter index.

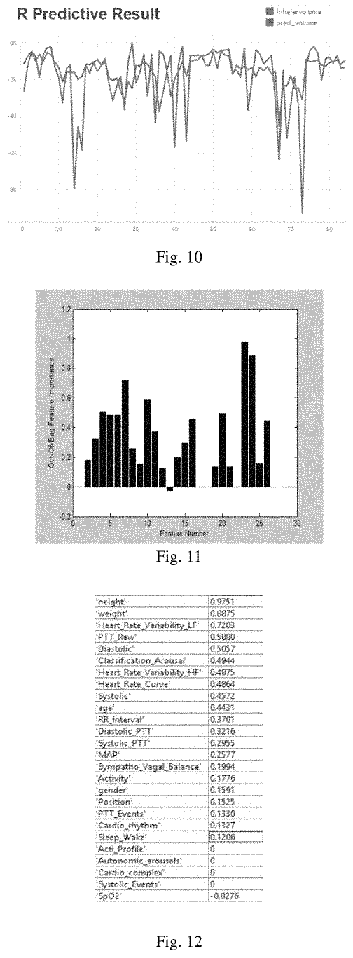

If the predictive result of the decision tree algorithm developed in R language is compared with the actual inhaling volume, as shown in FIG. 10, it is not difficult to see that the curve representing the prediction value and the curve representing the actual inhaling volume are relatively consistent roughly in the direction and trend of the curve, which indicating that the prediction is relatively accurate. However, it is worth noting that the deviation between the predictive result and the actual situation often occurs at the peaks and valleys of the curves. This is unavoidable, but it can be optimized by expanding the training set, optimizing judgment rules, and iterative calculation.

If the decision tree algorithm has been performed for 100 times, because there are 26 features, as shown in FIG. 11, the histogram evenly increases the mean square error (MSE) of all the trees for each attribute, and divides by the standard deviation of the trees. The larger the value of the bar chart, the more important this attribute is.

The matrix obtained by this method is not the same as before. As shown in FIG. 12, the second column of the matrix represents the average mean square error (MSE) of all the trees, divided by the standard deviation of each tree, but not just MSE. Similarly, the larger the value, the more degree of importance it is.

To sum up, it can be considered that height, weight, Heart Rate Variability LF, and PTT_RAW are the most relevant parameters, which is also consistent with people's common sense and logic. It is also generally believed that these parameters have a relatively great weight in predicting drug inhalation.

<Data Preprocessing>

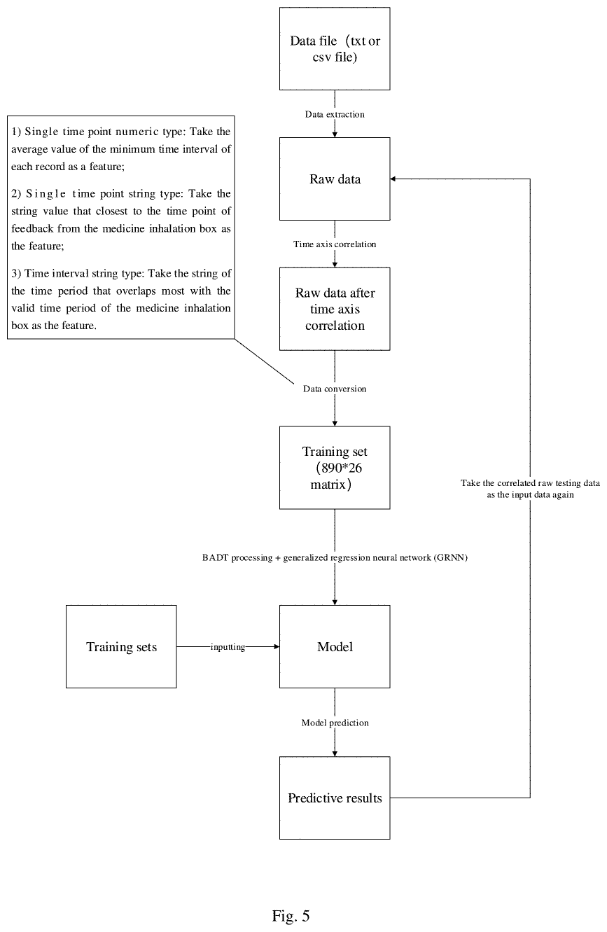

In addition, due to the large amount of information data, multiple dimensions of data, and complex relationships between data received from hardware devices, data preprocessing is required before entering the decision tree process. In other words, the raw data of the prediction algorithm needs to be organized and optimized In fact, this optimization is not just performed once, but continuously repeated. For the result of each algorithm (the result of the decision tree algorithm, that is, the output data of the decision tree), ETL (extract-transform-load) processing will be performed again, and then used as the input data of the decision tree algorithm again, so as to continuously iterate and continuously optimize the prediction accuracy of the algorithm. Data preprocessing of the entire system is shown in FIG. 5.

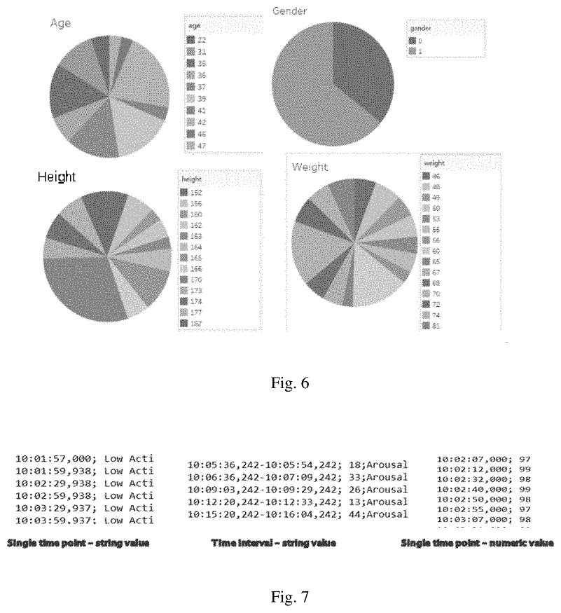

The obtained raw data, that is, Original .txt & .csv Data Files, contains a total of 26 data attributes, including representative and targeted physiological parameters such as Heart_Rate_Curve, Diastolic, SpO2, PTT, Systolic and so on. The coverage of the data source is also relatively wide, taking into account the various distribution of the audience, as shown in FIG. 6.

In the expected market research, it is preliminarily predicted that age, weight and height are relatively important for the predictive results. It is not difficult to see from the attribute map in FIG. 6 that the distribution is relatively uniform, the coverage is wide, and the general representation and validity of the data is great.

After reviewing the data overall, the time frame of reference for the data also needs to be determined. From the data sent by the hardware, there are two parts related to time—the relationship between time and inhalation condition sent by the medicine inhalation box, and the relationship between time and physiological parameters sent by the physiological detection device. A “bond” needs to be found to connect the two parts and to obtain the effect of inhalation condition on human physiology. In other words, the time interval between inhalation and physiological parameters needs to be found so that the two time axis can be correlated.

For this reason, the method adopted in the present application is to monitor the time interval of two feedbacks from the medicine inhalation box, and then take the smaller one as the required time interval t. In this way, if the feedback time of the medicine inhalation box is obtained as T, then it can be considered that the physiological parameter feedback in the (T−t, T+t) time is valid. In a few cases, it is also found that there is no physiological parameter information during this interval, taking into account the hardware response time and network transmission conditions, the time interval is selected to be extended outward for several seconds, such as 4-5 seconds, that is (T−t−4, T+t+4). If the physiological parameter information is still not detected, then it can be considered that this set of data is invalid and the two parts cannot be correlated.

After the time axis correlation is performed on the data (the data here refers to the inhaling volume sent by the medicine inhalation box and the physiological parameters sent by the physiological detection device), we start to extract and convert the inhaling volume sent by the medicine inhalation box and the physiological parameters sent by the physiological detection device after the time axis correlation. After a rough observation, we can see that the information of the sample data is basically divided into three types: single time point string format, time interval string format, and single time point numeric format (as shown in FIG. 7).

After understanding the data format, we can start to convert the data. Corresponding to three data types, the following three operations can be performed:

1) Single time point numeric type: take the average value of the minimum time interval of each record as a feature;
2) Single time point string type: take the string value that closest to the single time point of feedback from the medicine inhalation box as the feature;
3) Time interval string type: take the string of the time interval that overlaps most with the valid time interval of the medicine inhalation box as the feature, the valid time interval is (T−t, T+t) for example.

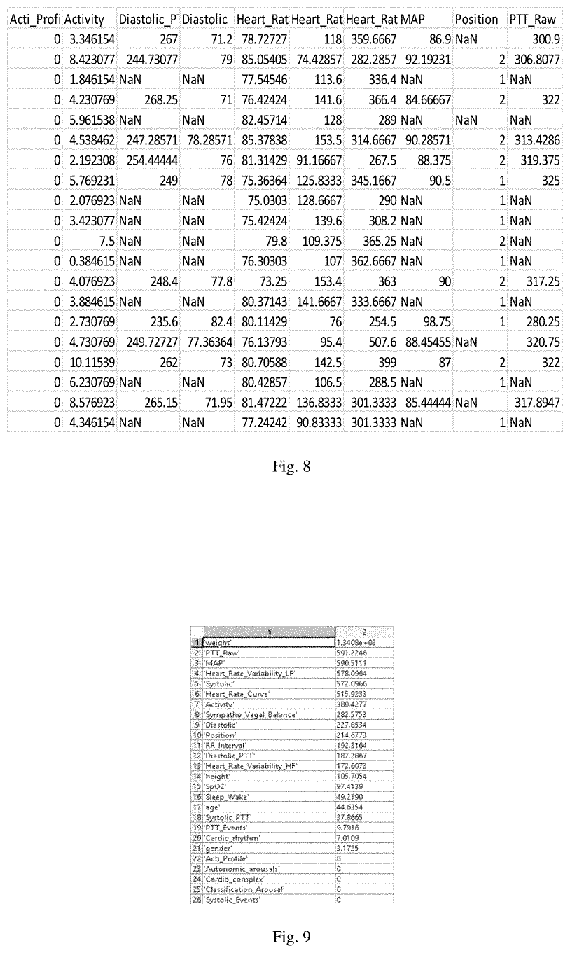

After these operations, all raw data that over 30 GB has been transformed, and an 890*26 matrix has been obtained, as shown in FIG. 8.

890 is the number of valid tuples, and 26 is the number of attribute dimensions. It greatly simplifies the workload of data processing and deletes a lot of unnecessary, incorrect, and invalid data. This data matrix is also the input information of the subsequent regression tree algorithm (that is, the input parameter matrix mentioned in the aforementioned classification and regression tree).

<Post Optimization—Neural Networks and BADT>

In addition, the above-mentioned decision tree model has generally met the design requirements, but there are still some problems in the details processing. In many cases, the binary tree nodes of the decision tree cannot meet the requirements, and the number of samples of a node is often greater than one. Therefore, the previous prediction is actually equivalent to taking the average of multiple sample predictions of a certain node. Of course, it would be harmless to just predict the general trend. However, considering the slight changes in the actual data, it is not enough to rely on a decision tree model only at present, so generalized regression neural network (GRNN) is introduced as the post-optimization.

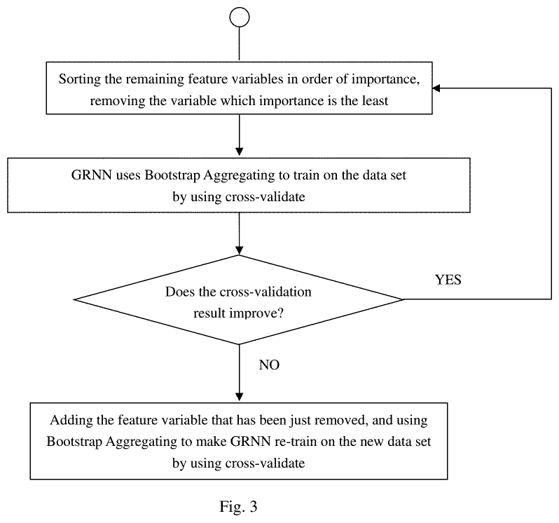

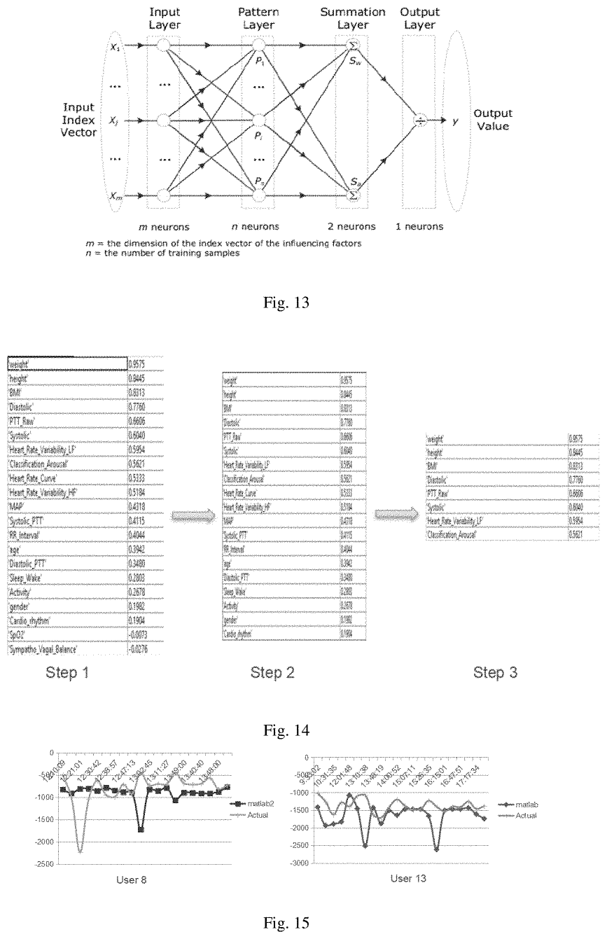

FIG. 13 shows the basic architecture of a generalized regression neural network. FIG. 3 shows a schematic flowchart of post-optimization of the output of a decision tree using a generalized regression neural network. The above-mentioned theoretical basis of network structure is mainly nonlinear regression analysis, and the network generally converges to an optimized regression with relatively large sample size accumulation. The structure is mainly divided into input layer, pattern layer, summation layer and output layer.
Input layer: The input is a vector, the dimension m is all the 26 attribute dimensions, and the transfer function is linear.
Pattern layer: The pattern layer is fully connected to the input layer. The number n of neurons in the layer is the number of samples, and the transfer function is a radial basis function.
Summation layer: There are only two nodes in the summation layer, the first node is the output sum of each pattern layer node, and the second node is the weighted sum of the expected result and each pattern layer node.
Output layer: The output is the second node in the summation layer divided by the first node.

The data processing process can be organized according to these four layers. The following simply represent the data processing process by mathematical formula: (Note: X is the network input variable, Xi is the learning sample corresponding to the ith neuron, σ is the standard deviation of the Gaussian function, and its value is artificially determined)

1) In the pattern layer, first, obtain the vector data of the input layer directly, the sample data is n, each neuron corresponds to a different sample, and the transfer function is:

$${p}_{i} = exp [ - \frac{{( X - {X}_{i} )}^{T} ( X - {X}_{i} )}{2 {a}^{2}} ] i = 1 , 2 , … , n$$

The output of neuron i is the square of Euclidean distance between the input variable and the corresponding sample before.
2) After entering the summation layer, there are only two neurons. The first neuron sums up as:

$${\sum }_{i = 1}^{n} exp [ - \frac{{( X - {X}_{i} )}^{T} ( X - {X}_{i} )}{2 {σ}^{2}} ]$$

Sum the outputs of the previous pattern layer, where the connection weight of the pattern layer and the neuron is 1, then the passing parameter is

$${S}_{D} = {\sum }_{i = 1}^{n} {P}_{i}$$

The second neuron sums up as:

$${\sum }_{i = 1}^{n} {Y}_{i} exp [ - \frac{{( X - {X}_{i} )}^{T} ( X - {X}_{i} )}{2 {σ}^{2}} ]$$

It represents the weighted summation of the neurons in the previous pattern layer. The connection weight value of the ith neuron in the pattern layer and the jth molecular summation neuron in the summation layer is the jth element in the ith output sample Yi. The passing parameters is:

$${S}_{N j} = {\sum }_{i = 1}^{n} {y}_{i} {P}_{i} j = 1 , 2 , … , k$$

3) Finally, to the output layer, the number of neurons in the output layer is equal to the dimension k of the output vector in the learning sample. Divide the outputs of the previous summation layers, where the output of the neuron j corresponds to the jth element of the predictive result Y, that is,

$${y}_{i} = \frac{{S}_{N j}}{{S}_{n}} j = 1 , 2 , … , k$$

4) After organizing and summarizing, it can be understood as the following formula:

$$Y ( x ) = \frac{{\sum }_{k = 1}^{N} {y}_{k} {e}^{{d}_{k} \text{/} 2 σ}}{{\sum }_{k = 1}^{N} {e}^{{d}_{k} \text{/} 2 σ}} , \\ {d}_{k} = {( x - {x}_{i} )}^{T} ( x - {x}_{i} )$$

In the formula, X is the input, Y is the prediction output, and dₖ is the square of the distance between the input X and the training sample Xi.

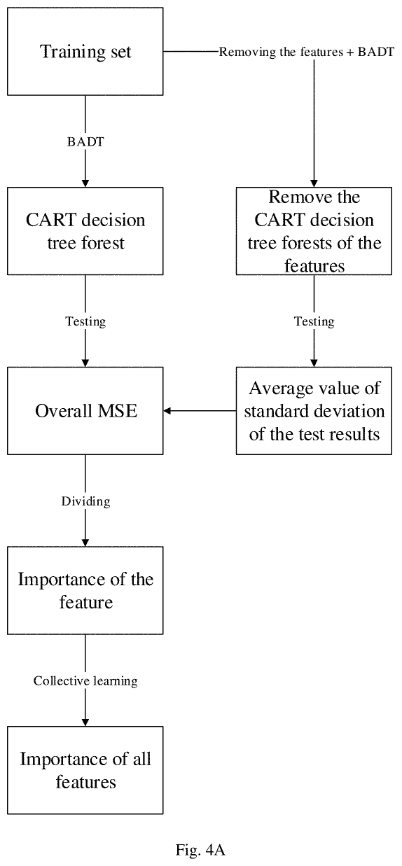

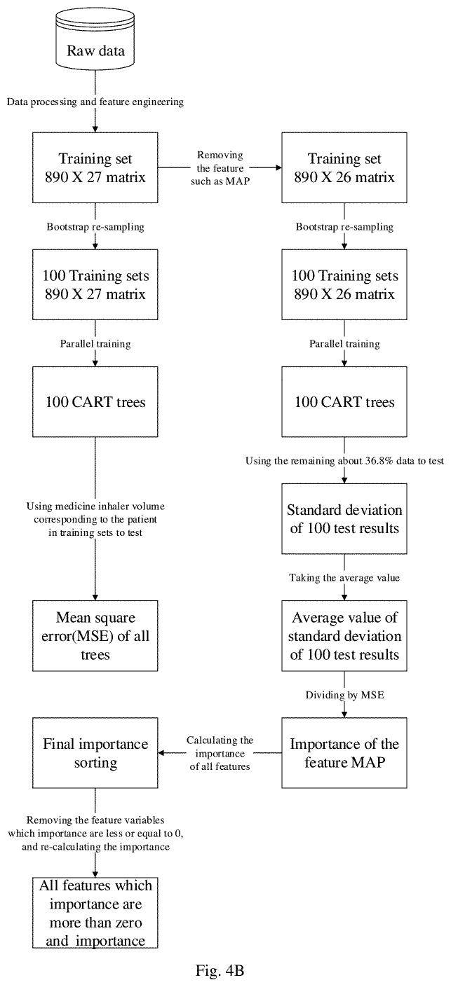

Through this method, the accuracy of prediction can be greatly improved, but there is still a problem: the generalized regression neural network does not allow the existence of illegal values such as null values, and the data also needs to be normalized in advance. Therefore, Bootstrap Aggregating Decision Tree (BADT) can also be used to process null data specifically. FIG. 4A-4B are schematic flowcharts of using BADT to process null data specifically to post-optimize the output of the decision tree, in which FIG. 4A shows the main process of BADT optimization, and FIG. 4B shows the detailed process of BADT optimization.

So far, the optimization process can also be simplified into the following steps:
(1) Build the BADT model and train the 26 physiological parameter variables to get the optimal model;
(2) Remove the parameter variables that have no influence or even negative influence on the inhaling volume from the medicine box from the results, and continue training;
(3) Repeat the above process until the remaining parameters all have positive influence, and they are sorted in descending order of importance.
(4) Input the variable data into the generalized regression neural network model for training. Every training can get a mean square error (MSE). Reduce the number of variables, find the minimum mean square error of each variable, so as to select the most important parameter variable.

After the optimization of GRNN and BADT, a new set of 26 attributes can be obtained, and the unimportant attributes are continuously removed. The final obtained resulting of optimization is shown in FIG. 14.

<Prediction Test>

When obtaining test data, dozens of groups of different ages, different genders, and different physical conditions were prepared. Among them, not much data is relatively stable, comprehensive, and valid. Take User 8 and User 13 as examples to verify the accuracy of the predictive results, as shown in FIG. 15.

The similarity between the data predictive results based on the matlab language application and the actual result is expected, the contrast in several points is relatively high, but the overall trend is relatively consistent.

The definition of accuracy here is: the percentage number obtained by first calculating the data that the error between the predicted and actual inhaling volume is within 50%, and then dividing by the total number of test sets.

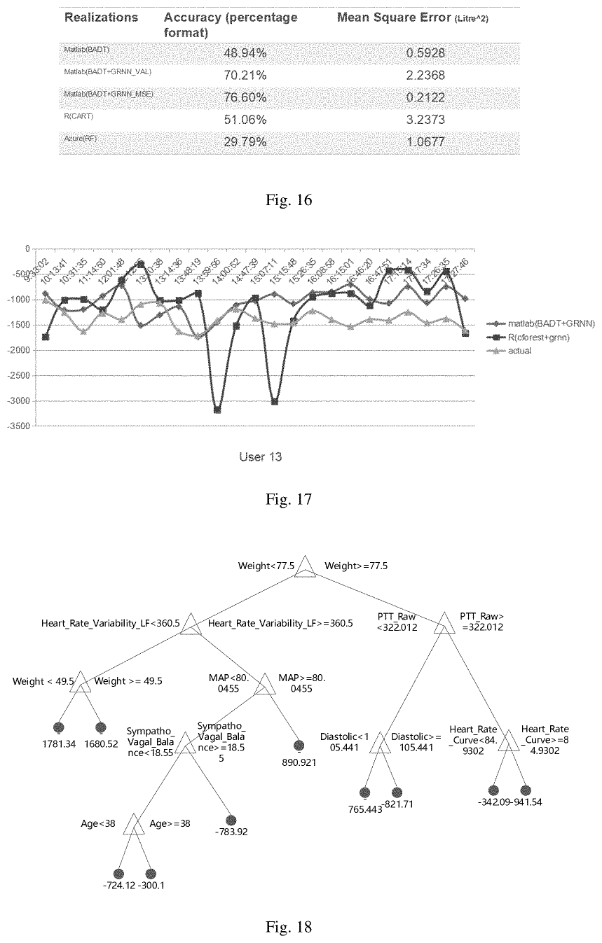

In FIG. 16, BADT represents Bootstrap Aggregating Decision Tree model, RF represents Random Forest model, and Azure is a machine learning model provided by Microsoft. Matlab (BADT+GRNN_VAL) and Matlab (BADT+GRNN_MSE) use the above-mentioned BADT and GRNN optimization models.

It is not difficult to see that the Matlab algorithm based on BADT and generalized regression neural network has relatively high accuracy, especially when the relatively small mean square error (MSE) is used as the measurement standard, the accuracy is improved to 76%. Compared with other algorithms, its performance is more excellent.

Taking User 13 as an example, as shown in FIG. 17, the curve representing the algorithm of BADT+GRNN agrees well with the curve representing the actual inhaling volume. It should be noted that when the same algorithm model is implemented in different programming languages and methods, the results will be different as well, for example, R language and Matlab, the machine learning algorithms have their own underlying programming difference, especially when the details are inspected, this difference will be more obvious.

In addition, the above-mentioned method of the present invention may be implemented by a storage medium installed in a computer device, and the storage medium may store instructions for performing the following steps: obtaining the dosage data and multiple human physiological parameter data of multiple testers as raw data; preprocessing the raw data to obtain input data as a training set; based on the input data, establishing a decision tree by classification and regression tree algorithm, which includes: generating a decision tree based on the feature extraction of the input data, and pruning the generated tree and selecting the optimal sub-tree by using the validation data set; receiving the user's human physiological parameter data, and predicting the required dosage according to the established decision tree. The above-mentioned computer device may be, for example, a server, a computer, or various types of mobile terminal. The storage medium may be, for example, a storage medium that stores instructions that can be read and executed by a computer device, and may be, for example, a disk-type storage medium or a storage medium built in the computer device.

Without departing from the essential characteristics of the present invention, the present invention may be embodied in various forms. Therefore, the embodiments in the present invention are used for illustration rather than limitation. Since the scope of the present invention is defined by the claims rather than the description, all changes that fall within the scope defined by the claims, or equivalents of the scope defined by the claims should be understood to be included in the claims.

What is claimed is:

1. A method for predicting the dosage based on human physiological parameters, comprising:
obtaining dosage data and multiple human physiological parameter data of multiple testers as raw data;
preprocessing the raw data to obtain input data as a training set;
based on the input data, establishing a decision tree by classification and regression tree algorithm, which includes:
generating the decision tree based on the feature extraction of the input data, and pruning the generated decision tree and selecting the optimal sub-tree by using a validation data set;
inputting a user's human physiological parameter data, and predicting the required dosage according to the established decision tree.

2. The method according to claim 1, further comprising:
using generalized regression neural network to post-optimize the output of the decision tree.

3. The method according to claim 1, further comprising:
using BADT to process null data specifically to post-optimize the output of the decision tree.

4. The method according to claim 1, wherein
the generation of the decision tree uses the Gini index to select the optimal feature, and to determine the optimal splitting point of the feature at the same time.

5. The method according to claim 1, the pruning comprising:
cutting off the sub-trees continuously from the bottom of the complete tree form of the decision tree; testing the sequence of the sub-trees on the independent verification data set by the cross-validation method, from which the optimal sub-tree is selected.

6. The method according to claim 1, the preprocessing comprising:
correlating the dosage data with the human physiological parameters on the time axis.

7. The method according to claim 1, the preprocessing further comprising:
processing the input data by ETL, and processing the output data of the decision tree by ETL again as input data, thereby continuously iterating.

8. A storage medium, which stores instructions that can be executed by a computer device and can be read by the computer device;
the instructions cause the computer device to perform the following steps:
obtaining dosage data and multiple human physiological parameter data of multiple testers as raw data;
preprocessing the raw data to obtain input data as a training set;
based on the input data, establishing a decision tree by classification and regression tree algorithm, which includes:
generating the decision tree based on the feature extraction of the input data, and pruning the generated decision tree and selecting the optimal sub-tree by using a validation data set;
receiving a user's human physiological parameter data, and predicting the required dosage according to the established decision tree.
